(19) United States
(12) Patent Application Publication
(10) Pub. No.: US 2021/0282980 A1
(43) Pub. Date: Sep. 16, 2021

(54) Diaper System

(71) Applicant: Benita Morris, Seattle, WA (US)

(72) Inventor: Benita Morris, Seattle, WA (US)

(21) Appl. No.: 16/817,266

(22) Filed: Mar. 12, 2020

(51) Int. Cl. A61F 13/42

(52) U.S. Cl. CPC A61F 13/42; A61F 2013/424

(57) ABSTRACT

A diaper system includes a diaper that may be worn thereby facilitating the diaper to absorb urine. A detection unit is coupled to the diaper and the detection unit detects the urine. The detection unit issues an alert when the detection unit detects the urine. Thus, the detection unit confirms that the diaper needs to be changed.

### BACKGROUND OF THE DISCLOSURE

### Field of the Disclosure

The disclosure relates to diaper devices and more particularly pertains to a new diaper device for issuing an alert when the diaper needs to be changed.

### SUMMARY OF THE DISCLOSURE

An embodiment of the disclosure meets the needs presented above by generally comprising a diaper that may be worn thereby facilitating the diaper to absorb urine. A detection unit is coupled to the diaper and the detection unit detects the urine. The detection unit issues an alert when the detection unit detects the urine. Thus, the detection unit confirms that the diaper needs to be changed.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective in-use view of a diaper system according to an embodiment of the disclosure.

FIG. 2 is a perspective view of an embodiment of the disclosure.

FIG. 3 is a top phantom view of an embodiment of the disclosure.

FIG. 4 is a bottom view of an embodiment of the disclosure.

FIG. 5 is a schematic view of an embodiment of the disclosure.

### DESCRIPTION OF THE PREFERRED EMBODIMENT

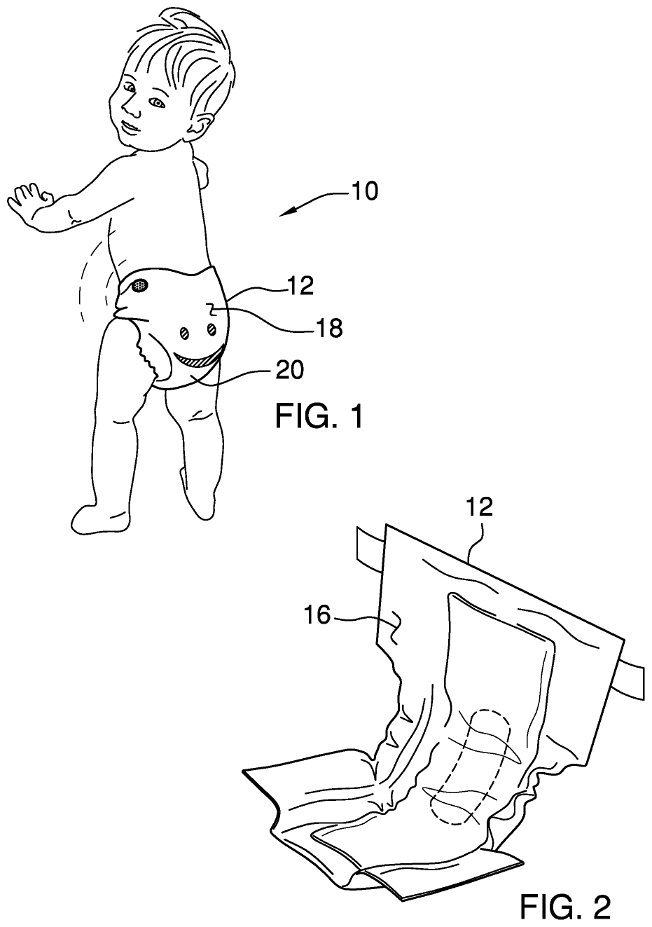

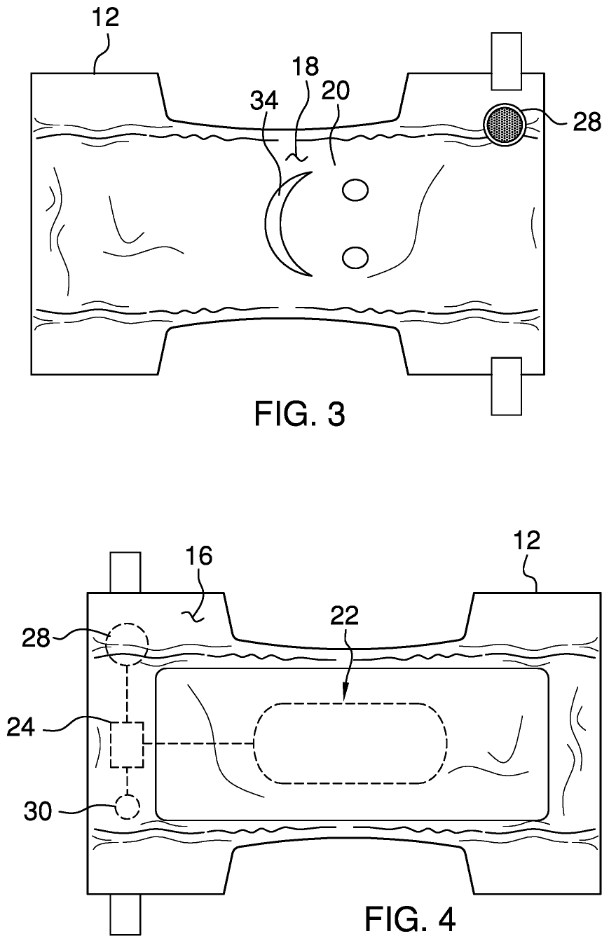

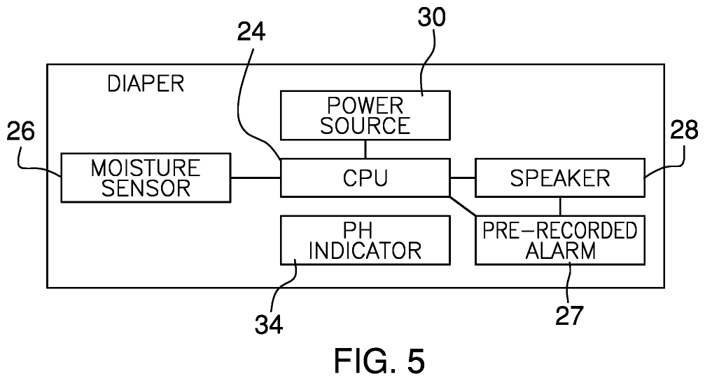

With reference now to the drawings, and in particular to FIGS. 1 through 5 thereof, a new diaper device embodying the principles and concepts of an embodiment of the disclosure and generally designated by the reference numeral 10 will be described.

As best illustrated in FIGS. 1 through 5, the diaper system 10 generally comprises a diaper 12 that may be worn thereby facilitating the diaper 12 to absorb urine 14. The diaper 12 has an inner surface 16, an outer surface 18 and a back side 20. The diaper 12 may be an absorbent diaper of any conventional design. Additionally, the diaper 12 may be worn by an infant or an adult.

A detection unit 22 is coupled to the diaper 12 such that the detection unit 22 may detect the urine 14. The detection unit 22 issues an alert when the detection unit 22 detects the urine 14. Thus, the detection unit 22 confirms that the diaper 12 needs to be changed. The detection unit 22 comprises a processor 24 that is positioned between the inner surface 16 and the outer surface 18 of the diaper 12. The processor 24 may comprise an electronic processor or the like. A moisture sensor 26 is positioned between the inner surface 16 and the outer surface 18 such that the moisture sensor 26 may detect the urine 14 in the diaper 12. The moisture sensor 26 is electrically coupled to the processor 24. The moisture sensor 26 may comprise an electronic moisture sensor or the like.

An electronic memory 27 is positioned between the inner surface 16 and the outer surface 18. The electronic memory 27 is electrically coupled to the processor 24. The electronic memory 27 stores data and the data may comprise a pre-recorded verbal phrase. The electronic memory 27 may comprise ROM memory or the like.

A speaker 28 is positioned on the outer surface 18 and the speaker 28 is electrically coupled to the processor 24. The speaker 28 emits an audible alarm when the moisture sensor 26 detects the urine 14 such that the speaker 28 confirms that the diaper 12 needs to be changed. The audible alarm may comprise a beep or the like or the audible alarm may comprise the pre-recorded verbal phrase stored in the electronic memory 27. A power supply 30 is positioned between the inner surface 16 and the outer surface 18. The power supply 30 is electrically coupled to the processor 24 and the power supply 30 comprises at least one battery 32.

A ph strip 34 is coupled to the outer surface 18 and the ph strip 34 is positioned on the back side 20 of the diaper 12. Thus, the ph strip 34 may come into contact with the urine 14. The ph strip 34 displays a vibrant color when the urine 14 contacts the ph strip 34. Thus, the ph strip 34 issues a visual alert to change the diaper 12. The ph strip 34 may be arranged to form an image of a smiley face or other image.

In use, the diaper 12 is worn. The speaker 28 emits the audible alarm and the ph strip 34 displays the vibrant color when the diaper 12 becomes soaked with the urine 14. The speaker 28 and the ph strip 34 inhibit the diaper 12 from being worn while the diaper 12 is soaked with the urine 14. The diaper 12 is removed and the diaper 12 is discarded when the diaper 12 becomes soaked with the urine 14.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A diaper system comprising:
a diaper configured to be worn thereby facilitating said diaper to absorb urine; and
a detection unit being coupled to said diaper wherein said detection unit is configured to detect the urine, said detection unit issuing an alert when said detection unit detects the urine wherein said detection unit is configured to confirm that the diaper needs to be changed.

2. The system according to claim 1, wherein:
said diaper has an inner surface and an outer surface; and
said detection unit comprises:
a processor being positioned between said inner surface and said outer surface of said diaper; and
a moisture sensor being positioned between said inner surface and said outer surface wherein said moisture sensor is configured to detect the urine in said diaper, said moisture sensor being electrically coupled to said processor.

3. The system according to claim 2, further comprising a speaker being positioned on said outer surface, said speaker being electrically coupled to said processor, said speaker emitting an audible alarm when said moisture sensor detects the urine wherein said speaker is configured to confirm that said diaper needs to be changed.

4. The system according to claim 2, further comprising a power supply being positioned between said inner surface and said outer surface, said power supply being electrically coupled to said processor, said power supply comprising at least one battery.

5. The system according to claim 1, further comprising:
said diaper having an outer surface; and
a ph strip being coupled to said outer surface wherein said ph strip is configured to come into contact with the urine, said ph strip displaying a vibrant color when the urine contacts said ph strip wherein said ph strip is configured to issue a visual alert to change the diaper.

6. A diaper system comprising:
a diaper configured to be worn thereby facilitating said diaper to absorb urine, said diaper having an inner surface and an outer surface; and
a detection unit being coupled to said diaper wherein said detection unit is configured to detect the urine, said detection unit issuing an alert when said detection unit detects the urine wherein said detection unit is configured to confirm that the diaper needs to be changed, said detection unit comprising
a processor being positioned between said inner surface and said outer surface of said diaper,
a moisture sensor being positioned between said inner surface and said outer surface wherein said moisture sensor is configured to detect the urine in said diaper, said moisture sensor being electrically coupled to said processor,
a speaker being positioned on said outer surface, said speaker being electrically coupled to said processor, said speaker emitting an audible alarm when said moisture sensor detects the urine wherein said speaker is configured to confirm that said diaper needs to be changed,
a power supply being positioned between said inner surface and said outer surface, said power supply being electrically coupled to said processor, said power supply comprising at least one battery, and
a ph strip being coupled to said outer surface wherein said ph strip is configured to come into contact with the urine, said ph strip displaying a vibrant color when the urine contacts said ph strip wherein said ph strip is configured to issue a visual alert to change the diaper.
